(19) United States
(12) Patent Application Publication
(10) Pub. No.: US 2019/0223727 A1
(43) Pub. Date: Jul. 25, 2019

(54) SYSTEM, DEVICE, AND METHOD FOR DETERMINING A TOTAL CONTENT OF A TARGET CHEMICAL IN A MICROLITER SAMPLE

(71) Applicant: Case Western Reserve University, Cleveland, OH (US)

(72) Inventors: Miklos Gratzl, Cleveland, OH (US); Tamas Cserfalvi, Cleveland, OH (US); Mihailo Rebec, Cleveland, OH (US)

(21) Appl. No.: 16/340,189

(22) PCT Filed: Oct. 10, 2016
(86) PCT No.: PCT/US2016/056219

§ 371 Date: Apr. 8, 2019

(51) Int. Cl. A61B 5/00; A61B 5/145

(52) U.S. Cl. CPC A61B 5/0055; A61B 5/4266; A61B 5/14514

(57) ABSTRACT

One aspect of the present disclosure relates to a handheld device that can be used to perform a screening or diagnostic test. The handheld device can include a disposable microsampler unit, an analysis unit, a controller unit, and an output unit. The disposable microsampler unit can collect 10 microliters or less of a sample. The analysis unit can include two electrodes that can apply alternating periods of coulometry and potentiometry to the sample to determine a total content of a target chemical in the sample. During the coulometry period, the two electrodes are a working electrode and a counter electrode, and during the potentiometry period the two electrodes are an indicator electrode and a reference electrode. The controller unit can control the sequence of coulometry and potentiometry. The output unit can display the total content of the target chemical in the sample.

### TECHNICAL FIELD

The present disclosure relates generally to determining a total content of a target chemical in a sample and, more specifically, to systems, devices, and methods that can determine the total content of the target chemical in a small (e.g., microliter) sample volume.

### BACKGROUND

Cystic fibrosis is a chronically debilitating autosomal recessive genetic disorder that affects the respiratory, gastrointestinal, and reproductive systems. Indeed, cystic fibrosis is the most prevalent life-shortening, childhood-onset, hereditary disease among white children. Cystic fibrosis is characterized by deficient chloride transport. For example, in the lungs, the deficient chloride transport leads to the production of abnormally thick mucus, which causes airway obstruction, neutrophil-dominated inflammation, and recurrent and progressive pulmonary infections.

Early diagnosis of cystic fibrosis, before symptoms appear, is beneficial for successful management of cystic fibrosis. Specifically, early diagnosis enables treatment to reduce the frequency and severity of pulmonary, gastrointestinal, and cognitive disorders and improve life expectancy. Accordingly, all 50 states mandate a newborn screening (NBS) blood test to identify carriers of cystic fibrosis. However, this blood test is only the first step of screening and cannot be used for diagnosis and has a very high rate of false positives and a considerable rate of false negatives. If a newborn screens as positive in the blood test and a further genetic or IRT test, the newborn must undergo a sweat chloride test, the gold standard for cystic fibrosis diagnosis. However, the sweat chloride test can only be given to older babies that can produce the amount of sweat necessary for conduction of the test. This means a delay of weeks and sometimes months before cystic fibrosis is diagnosed, in which these babies often begin to show the irreversible damage due to cystic fibrosis. Additionally, specialized expertise is necessary to perform the sweat test, so the sweat test is only available in a few accredited centers in each state. Moreover, a positive screening result can cause anxiety, stress, and even depression in parents until their newborn is diagnosed as positive or negative with the sweat test.

### SUMMARY

The present disclosure relates generally to determining a total content of a target chemical in a sample and, more specifically, to systems, devices, and methods that can determine the total content of the target chemical in a small (e.g., microliter) sample volume.

In one aspect, the present disclosure can include a handheld device that can perform a screening or diagnostic test. The handheld device can include a disposable microsampler unit configured to collect 10 microliters or less of a sample. The handheld device can also include an analysis unit comprising two electrodes configured to apply alternating periods of coulometry and potentiometry to the sample to determine a total content of a target chemical in the sample. During the coulometry period, the two electrodes act as a working electrode and a counter electrode, while during the potentiometry period, the two electrodes act as an indicator electrode and a reference electrode. The handheld device can also include a controller unit configured to control the sequence of coulometry and potentiometry; and an output unit configured to display the total content of the target chemical in the sample.

In another aspect, the present disclosure can include a method for performing a screening or diagnostic test. Ten microliters or less of a sample can be collected in a capillary shaped with a pulled upper end. The sample can be transferred from the capillary into an analysis unit. Within the analysis unit, the sample can be diluted with a buffer, and a sequence can be performed comprising alternating periods of coulometry and potentiometry on the diluted sample to determine a total content of a target chemical in the sample. The analysis unit can include two electrodes such that in the coulometry period, the two electrodes are a working electrode and a counter electrode, and during the potentiometry period, the two electrodes are an indicator electrode and a reference electrode. The total content of the target chemical in the sample can be displayed as an output.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a block diagram illustrating an example of a system that can determine the total content of a target chemical in a small sample volume, according to an aspect of the present disclosure;

FIG. 2 shows a block diagram of an example configuration of a disposable portion of the system in FIG. 1;

FIG. 3 shows a block diagram of the example configuration in FIG. 2 with a sample creation unit to facilitate gathering the sample;

FIG. 4 shows a process flow diagram of a method for determining the total content of a target chemical in a small sample volume, according to another aspect of the present disclosure; and

FIG. 5 shows a process flow diagram of an example method for analyzing the total content of the target chemical in the sample that can be employed within the method in FIG. 4.

### DETAILED DESCRIPTION

### I. Definitions

Unless defined otherwise, all technical and scientific terms used herein have the same meaning as is commonly understood by one of skill in the art to which the present disclosure pertains.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure.

The sequence of operations (or acts/steps) is not limited to the order presented in the claims or figures unless specifically indicated otherwise.

As used herein, the term “potentiometry” can refer to the non-destructive, passive measurement of the potential of a sample. One electrode is called the reference electrode and has a constant potential, while the other electrode, called the indicator electrode, has a potential that changes with the composition of the target chemical in the sample. In other words, the difference in potential between the two electrodes gives an assessment of the composition of target chemical in the sample.

As used herein, the term “coulometry” can refer to the measurement of a current over time. For example, coulometry can relate to using applied current or potential to completely convert a target chemical from one oxidation state to another. The total current passed is measured directly or indirectly to determine the number of electrons passed. Knowing the number of electrons passed can indicate the concentration of the target chemical or, when the concentration is known, the number of electrons transferred in the redox reaction. Coulometry uses two electrodes: a working electrode and a counter electrode.

As used herein, the term “sample”, can refer to a specimen taken for scientific testing or analysis. Different examples of samples can include a biological product (e.g., blood, urine, tissue, sweat, etc.), water, soil, an agricultural product, and the like. In some examples, the sample can have a volume of 10 microliters or less. In other examples, the sample can have a volume of 5 microliters or less. In still other examples, the sample can have a volume of 3 microliters or less. In further examples, the sample can have a volume of between 1 and 2 microliters.

As used herein, the term “absolute measurement” can refer to a measure expressed in a number the same as data recorded.

As used herein, the term “target chemical” can refer to an analyte within the sample. In other words, the target chemical is being identified and/or measured within the sample.

As used herein, the term “substantial accuracy” can refer to a complete (e.g., 100%) or partial (e.g., less than 100%, such as about 99%, about 95%, about 93%, about 90%, about 80%, about 70%, about 60%, or less than about 50%) lack of false negative screening tests or diagnostic tests and/or false positive screening tests or diagnostic tests.

As used herein, the term “quantitative” can refer to a quantity that can be measured. For example, the results of a quantitative measurement can include numerical data.

As used herein, the term “early diagnosis” can refer to a diagnosis achieved from diagnostic test performed on an infant less than 45 days old. As another example, the diagnostic test can be performed on the infant less than 30 days old. As a further example, the diagnostic test can be performed on the infant less than 15 days old. As another example, the diagnostic test can be performed on the infant less than 8 days old.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

As used herein, the term “medical professional” can be used to refer to an individual who provides care to the patient (e.g., the medical professional can administer one or more medical tests to the patient). The medical professional can be, for example, a doctor, a physician's assistant, a student, a nurse, a caregiver, or the like.

### II. Overview

The present disclosure relates generally to determining a total content of a target chemical in a sample (e.g., a biological/physiological sample, a water sample, a soil sample, an agricultural product sample, etc.). More specifically, the present disclosure relates to systems, devices, and methods that can determine the total content of the target chemical in a small sample volume (e.g., microliter). For example, the target chemical can be chloride, and the absolute amount of chloride in a sweat sample can be determined and used for the screening and/or diagnosis of cystic fibrosis. As another example, the target chemical can be iodide or bromide, and the absolute amount of iodide or bromide in a microsample can be determined to aid the chemical and/or pharmaceutical industry. Additionally, as a further example, the target chemical can include one or more proteins, and the protein contents of small samples can be determined via Ag+ based precipitation of proteins in clinical diagnostics and/or research. Other target chemicals can be identified in waste water samples and/or in samples taken from other environmental contexts.

As one example, the systems, devices, and methods described herein can be used to detect the amount of chloride in a sweat sample to facilitate the screening and/or diagnosis of cystic fibrosis. While early diagnosis of cystic fibrosis, before symptoms appear, is beneficial for successful management of cystic fibrosis, enabling treatment to reduce the frequency and severity of pulmonary, gastrointestinal, and cognitive disorders and improve life expectancy and all 50 states mandate a newborn screening (NBS) blood test to identify carriers of cystic fibrosis, the ultimate diagnosis can only occur with the gold standard sweat chloride test. However, the sweat test requires a volume of sweat that cannot be produced by a newborn. Instead, the sweat chloride test can only be given to older babies that can produce the amount of sweat necessary for conduction of the test, causing a delay of weeks and sometimes months before cystic fibrosis is diagnosed, in which these babies often begin to show the irreversible damage due to cystic fibrosis. Additionally, specialized expertise is necessary to perform the sweat test, so the sweat test is only available in a few accredited centers in each state. Moreover, a positive screening result can cause anxiety, stress, and even depression in parents until their newborn is diagnosed as positive or negative with the sweat test. These problems clearly show the need for an early sweat chloride test (using a small volume of sweat—10 microliters or less—that can be produced by a newborn) that would be accurate enough for reliable diagnosis, and that could be performed at any clinic or at the point of care. This would have a major positive impact on cystic fibrosis patients' quality of life and life expectancy, the importance of which is hard to over-emphasize. The societal impacts would also be significant due to the reduction of the high costs of continuous healthcare required during the entire lifetime of a cystic fibrosis patient who was not diagnosed early enough to prevent irreversible damage to occur before treatment could have begun.

### III. Systems

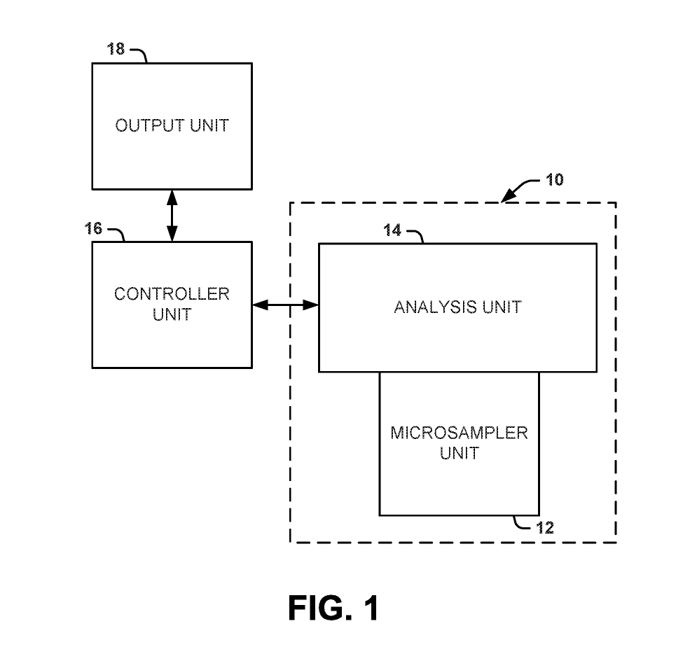

One aspect of the present disclosure, as shown in FIG. 1, includes a system that can determine the total content of the target chemical in a small (e.g., microliter) sample. For example, the system can employ just two electrodes to perform alternating periods of coulometry and potentiometry to determine the total concentration of the target chemical. Described herein is an example where the target chemical is chloride and the sample is sweat for screening and/or diagnosis of cystic fibrosis. However, different examples of samples can include a biological product (e.g., blood, urine, tissue, etc.), water, soil, an agricultural product, and the like. In some instances, the system can be embodied in a handheld device. The handheld device can be used at a clinic or other point of care. For example, the handheld device does not require specialized training to use. Additionally, the handheld device is small and portable compared to other devices that employ coulometry.

The system can include a disposable unit 10 that can include a microsampler unit 12 and at least a portion of an analysis unit 14. The system can also include a non-disposable portion. The non-disposable portion can include a non-disposable portion of the analysis unit 14. The non-disposable portion can also include a controller unit 16 that can control data collection by the analysis unit 14 and process data collected by the analysis unit 14. The controller unit 16 can include at least a hardware processor to facilitate the control and the data processing. In some instances, the controller unit 16 can be programmed to perform the control and the data processing. The system can also include an output unit 18 that can display results of the processing in a human-comprehensible manner. The display can include an indication related to the total content of the target chemical in the sample. The indication can be easy to read so that a medical professional without specialized skills can administer the test and provide the recording. For example, the total content of the target chemical can be matched to a standard list of concentrations corresponding to diseased and non-diseased patients. As another example, the display can include an indication of whether the patient is diseased or non-diseased.

The system can perform the test for cystic fibrosis screening and/or diagnosis with substantial accuracy on a young subject using a small volume of sweat that can be produced by an infant or even a newborn baby, allowing the test to be performed before permanent damage due to cystic fibrosis occurs. For example, the test can be performed on an infant less than 45 days old. As another example, the test can be performed on the infant less than 30 days old. As a further example, the test can be performed on the infant less than 15 days old. As another example, the test can be performed on the infant less than 8 days old. Thus, the diagnosis or screening can enable treatment if the patient does exhibit cystic fibrosis to reduce the frequency and severity of pulmonary, gastrointestinal, and cognitive disorders and improve life expectancy.

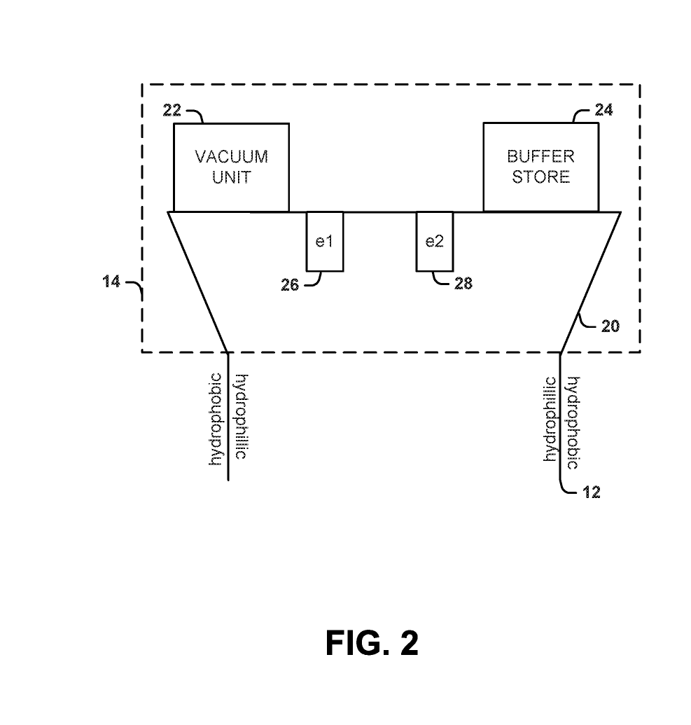

The disposable unit 10 is shown in greater detail in FIG. 2 (however, portions of FIG. 2 are not disposable). The disposable unit 10 generally includes the microsampler unit 12 and a portion of the analysis unit 14. For example, the disposable portion of the analysis unit 14 can include a reagent reservoir 20 and the non-disposable portion of the analysis unit 14 can include a vacuum unit 22 and a buffer store 24. Electrodes (e1 26 and e2 28) can be disposable, non-disposable, or a combination of disposable and non-disposable.

The microsampler unit 12 can be shaped and configured to collect a small volume of the sample. In some examples, the sample can have a volume of 10 microliters or less. However, in other examples, the sample can have a volume of 5 microliters or less. In still other examples, the sample can have a volume of 3 microliters or less. In further examples, the sample can have a volume of between 1 and 2 microliters.

The microsampler unit 12 can include a microcapillary that is sized and dimensioned to facilitate collection of the sample. The volume of sample that can be collected by the microcapillary can be based on the geometry (length and internal diameter) of the microcapillary. To facilitate collection of the small volume of the sample, the capillary can be shaped with a pulled upper end and can have a hydrophobic outer surface and hydrophilic inner surface. The cross-sectional surface where the sampling happens can be hydrophobic, as well. One example of such a microcapillary includes a fused silica capillary engineered with a hydrophobic outer surface. Another example of such a microcapillary includes a borosilicate glass capillary engineered with a hydrophobic outer surface. Upon contacting a quantity of the sample, a small volume of the sample can fill the microcapillary. For example, the filling can be due at least in part to capillary action. The sample can be transferred from the microcapillary to the reagent reservoir 20. One example of the transferring can be through aspiration.

A vacuum unit 22 interfaces with the reagent reservoir 20. The reagent reservoir 20 can hold a volume of fluid, including at least the sample and a buffer. In some instances, the reagent reservoir 20 can hold a volume of 500 microliters of fluid or more. In other examples, the reagent reservoir 20 can hold a volume of 400 microliters of fluid or more. However, the reagent reservoir 20 can be smaller than 400 microliters. The vacuum unit 22 can apply a pressure to facilitate the transferring of the sample from the capillary into the reagent reservoir 20. As an example, the vacuum unit 22 can include a mild vacuum (e.g., exerting a low negative pressure, such as −50 Torr), which can be provided by a miniature battery-driven motor-pump. However, the vacuum unit 22 can include any source of suction that can facilitate the transferring of the sample from the capillary into the reagent reservoir 20.

Once the sample is entirely within the reagent reservoir 20, the buffer store 24 can add a buffer to dilute the sample. The buffer store 24 can include an amount of buffer sufficient to perform a number of tests (e.g., 50 or more). In some instances, the buffer store 24 can be refillable with the buffer. One example of a buffer that can be added is a phosphate buffered saline (PBS) buffer. However, the buffer can be any type of buffer that can be added to dilute the sample. For example, a known volume of buffer can be used to dilute the sample. The buffer can have a volume that is between 5 and 15 times greater than the volume of the sample. In some examples, the buffer can have a volume that is between 8 and 12 times greater than the volume of the sample. In other examples, the buffer can have a volume that is between 9 and 11 times greater than the volume of the sample. The vacuum unit 22 can apply the low vacuum to mix, stir, agitate, etc. the buffer and sample so that the sample is evenly distributed within the buffer. In some instances, the mixing can be applied continuously. As an example, the vacuum unit 22 can generate small air bubbles entering from the microcapillary to agitate the buffer-sample solution. In another example, the vacuum unit 22 can include a piezo vibrator to sonicate the buffer-sample solution.

The analysis unit 14 can include two electrodes (e1 26 and e2 28) configured to perform alternating periods of coulometry (coulometric titration, constant current injected into the diluted sample) and potentiometry (potential measuring, open circuit potential measures the voltage difference between the two electrodes). The alternating periods can be established by the controller unit 16 as shown in FIG. 1. Advantageously, coulometry needs no calibration and is an approved technique for determining the concentration of chloride in sweat. Potentiometry involves no interference with coulometry. For example, potentiometry can be used for generation of silver ions from an electrode, and the silver ions can be consumed by chloride ions—this guarantees that all current flow is used for silver generation alone. However, either AC impedance in the 100 Hz range for addressing Faradaic resistance with little or no net current flow can be used additionally or alternatively to potentiometry. Alternatively, square wave amperometry in the 1 Hz range for assessing diffusion current, with little or no net current can be used additionally or alternatively to potentiometry. For example, the additive net amperometry current can be integrated for the correction of the coulometry current.

During the coulometry period, one electrode (e1 26) is a working electrode and the other electrode (e2 28) is a counter electrode. During the potentiometry period, one electrode (e1 26) is an indicator electrode and the other electrode (e2 28) is a reference electrode. However, the two electrodes (e1 26 and e2 28) are physically the same electrodes during both coulometry and potentiometry, although the electrodes may have different functions. It will be understood that either e1 26 or e2 28 can be the working electrode or the counter electrode and, independently, either the indicator electrode or the reference electrode. This is in contrast to traditional systems that use four electrodes, two for coulometry and two for potentiometry.

For example, the alternating periods of coulometry and potentiometry can include a period of current injection followed by a potential measurement. The periods can be defined by the controller unit 16, as shown in FIG. 1. As an example, in situations where the target chemical is chloride, e1 26 can be a silver electrode and e2 can be a gold, platinum, iridium, or palladium electrode. Indeed, e2 can be configured for current injection and its potential becomes pH dependent with no current injection. At least one of e1 and e2 can be a thin-film electrode. The thin film electrode can provide cheap but precise fabrication with negligible material cost.

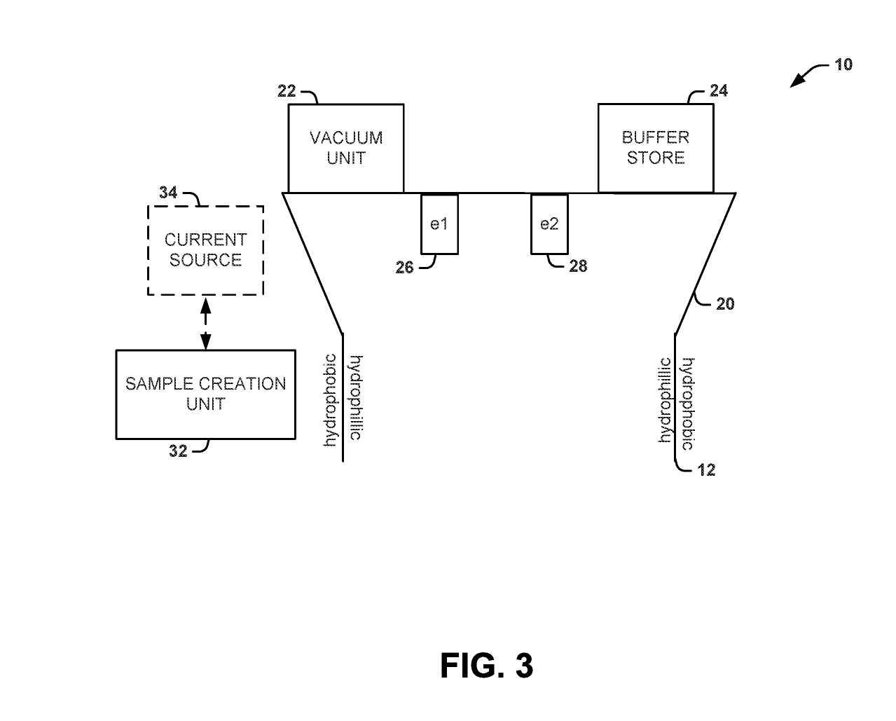

FIG. 3 shows an alternate configuration of the disposable portion. For example, the disposable portion can include a sample creation unit 32 to facilitate production of the sample. In some instances, the sample creation unit 32 can be coupled to a (non-disposable) current source 34 to facilitate the sample production. For example, the sample creation unit 32 can include an iontophoresis unit, which can be coupled to the current source 34 to cause sweat to be generated. In some examples, the current source 34 can also be connected to at least a portion of the analysis unit 14.

### IV. Methods

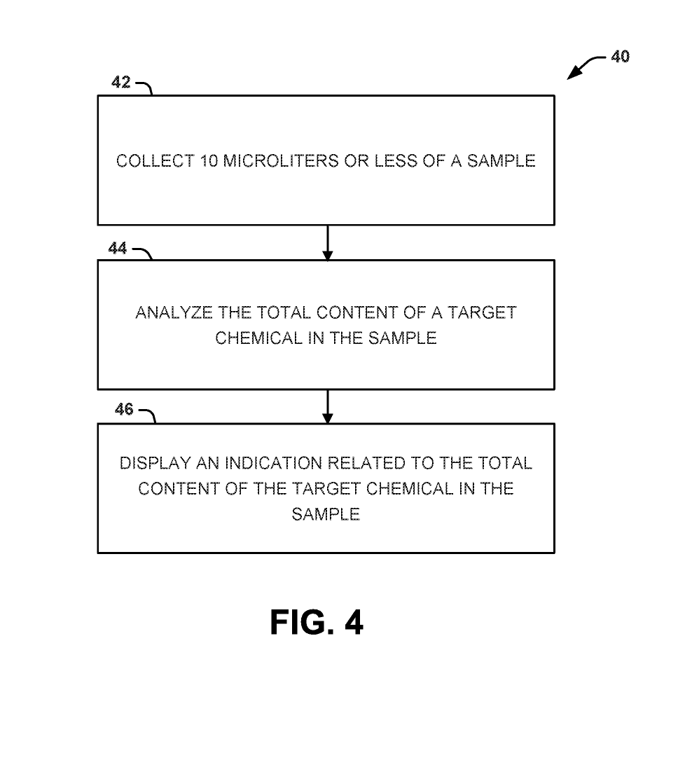

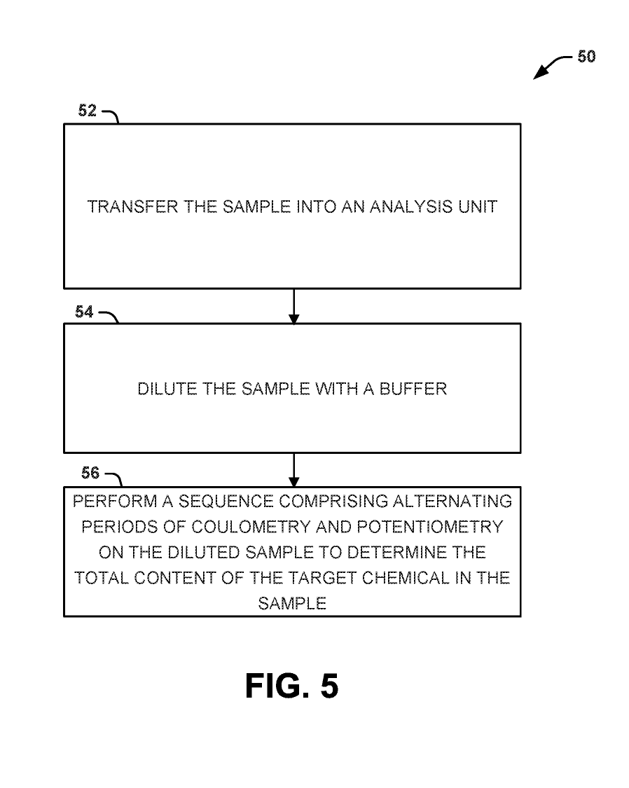

Another aspect of the present disclosure can include methods for determining the total content of the target chemical in a small (e.g., microliter) sample. The methods can employ a system that is identically or similarly configured to the system shown in any one of FIGS. 1-3 to perform the determination. In some examples, the system can be embodied as a handheld device with a disposable unit 10, as described above. One example of a method 40 that can determine the total content of the target chemical in a small sample is shown in FIG. 4. Another example of a method 50 that can analyze the total content of the target chemical in the sample is shown in FIG. 5.

The methods 40 and 50 of FIGS. 4 and 5, respectively, are illustrated as process flow diagrams with flowchart illustrations. For purposes of simplicity, the methods 40 and 50 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 40 and 50.

Referring to FIG. 4, another aspect of the present disclosure can include a method 40 for determining the total content of the target chemical in a small sample. At 42, the sample with a volume of 10 microliters or less can be collected. However, in other examples, the sample can have a volume of 5 microliters or less. In still other examples, the sample can have a volume of 3 microliters or less. In further examples, the sample can have a volume of between 1 and 2 microliters. Different examples of samples can include a biological product (e.g., blood, urine, tissue, sweat, etc.), water, soil, an agricultural product, and the like. In some instances, production of the sample can be triggered by a sample creation unit (like an iontophoresis unit).

At 44, the total content of the target chemical in the sample can be analyzed. The total content of the target chemical can be determined with substantial accuracy. In some instances, the method 40 can be used to determine the total content of chloride in a sweat sample to facilitate the screening and/or diagnosis of cystic fibrosis. Indeed, the screening and/or diagnosis of method 40 can be performed earlier than traditional methods, requiring a far smaller volume of sweat. For example, the method 40 can be performed on an infant less than 45 days old. As another example, the method 40 can be performed on the infant less than 30 days old. As a further example, the method 40 can be performed on the infant less than 15 days old. As another example, the method 40 can be performed on the infant less than 8 days old. The method 40 can be performed on a small volume of sweat that can be produced by an infant or even a newborn baby, allowing the test can be performed before permanent damage due to cystic fibrosis occurs. Thus, the diagnosis or screening can enable treatment if the patient does exhibit cystic fibrosis to reduce the frequency and severity of pulmonary, gastrointestinal, and cognitive disorders and improve life expectancy.

At 46, an indicated related to the total content of the target chemical in the sample can be displayed. The indication can be easy to read so that a medical professional without specialized skills can administer the test and provide the recording. For example, the total content of the target chemical can be matched to a standard list of concentrations corresponding to diseased and non-diseased patients. As another example, the display can include an indication of whether the patient is diseased or non-diseased.

Referring now to FIG. 5, illustrated is one example of a method 50 for analyzing the total content of the target chemical in the sample. At 52, the sample can be transferred into an analysis unit. For example, a capillary can contact a sample. For example, the capillary can contact a drop of sweat on a patient's skin. The capillary can have a hydrophilic interior and a hydrophobic exterior and tip to facilitate the sample entering the capillary. When the target volume (e.g., 10 microliters) of the sample fills the capillary (e.g., through capillary action), the sample is transferred into the analysis unit. For example, the transferring can be facilitated using a low vacuum (or other source or suction). In other words, the sample can be aspirated from the capillary into a reagent reservoir inside the analysis unit.

Once the sample is entirely within the reservoir, at 54, the sample can be diluted with a buffer. For example, a known volume of buffer can be used to dilute the sample. The buffer can have a volume that is between 5 and 15 times greater than the volume of the sample. In some examples, the buffer can have a volume that is between 8 and 12 times greater than the volume of the sample. In other examples, the buffer can have a volume that is between 9 and 11 times greater than the volume of the sample. The low vacuum (or other source of suction) can be used to stir or mix the diluted sample within the analysis unit. In some instances, the stirring or mixing can be continuous.

At 56, the total content of the target chemical in the sample can be determined by performing a sequence of alternating periods of coulometry and potentiometry on the diluted sample. The sequence can be a time sequence that includes a coulometry on time, a coulometry off time, a potentiometry on time, and a potentiometry off time. The analysis unit can have two electrodes, such that during the coulometry period, the two electrodes are a working electrode and a counter electrode, and during the potentiometry period, the two electrodes are an indicator electrode and a reference electrode. In some instances, at least one of the two electrodes can be implemented as a thin film.

From the above description, those skilled in the art will perceive improvements, changes and modifications. Such improvements, changes and modifications are within the skill of one in the art and are intended to be covered by the appended claims.

What is claimed is:

1. A handheld device comprising:
a disposable microsampler unit configured to collect 10 microliters or less of a sample;
an analysis unit comprising two electrodes configured to apply alternating periods of coulometry and potentiometry to the sample to determine a total content of a target chemical in the sample, wherein in the coulometry period the two electrodes operate as a working electrode and a counter electrode, and during the potentiometry period the two electrodes operate as an indicator electrode and a reference electrode;
a controller unit configured to control the sequence of coulometry and potentiometry; and
an output unit configured to display the total content of the target chemical in the sample.

2. The handheld device of claim 1, further comprising a disposable iontophoresis unit configured to trigger production of the sample by iontophoresis.

3. The handheld device of claim 2, wherein the disposable iontophoresis unit is configured to connect to a current source to facilitate the iontophoresis.

4. The handheld device of claim 1, wherein the disposable microsampler unit comprises a capillary shaped with a pulled upper end with a hydrophilic interior to facilitate collection of the sample and a hydrophobic exterior.

5. The handheld device of claim 4, further comprising a low vacuum to aspirate the sample from the capillary and into the analysis unit.

6. The handheld device of claim 5, wherein the low vacuum continuously mixes the sample within the analysis unit.

7. The handheld device of claim 1, wherein the analysis unit comprises a disposable reagent reservoir to hold the sample in a diluting buffer for analysis.

8. The handheld device of claim 5, wherein the disposal reagent reservoir comprises a volume of 500 microliters or less.

9. The handheld device of claim 1, wherein one of the electrodes comprises silver and the target chemical comprises chloride.

10. The handheld device of claim 9, wherein the other electrode is configured for current injection and its potential becomes pH dependent when no current injection.

11. The handheld device of claim 10, wherein the reference electrode comprises gold, platinum, iridium, or palladium.

12. The handheld device of claim 1, wherein at least one of electrodes comprises a thin-film electrode.

13. The handheld device of claim 1, wherein the disposable microsampler unit configured to collect 3 microliters or less of the sample.

14. A method comprising:
collecting 10 microliters or less of a sample in a capillary shaped with a pulled upper end;
transferring the sample from the capillary into an analysis unit;
diluting the sample with a buffer in the analysis unit;
performing a sequence comprising alternating periods of coulometry and potentiometry on the diluted sample in the analysis unit to determine a total content of a target chemical in the sample,
wherein the analysis unit comprises two electrodes such that during the coulometry period, the two electrodes are a working electrode and a counter electrode, and during the potentiometry period, the two electrodes are an indicator electrode and a reference electrode, and
displaying the total content of the target chemical in the sample.

15. The method of claim 14, wherein the capillary comprises a hydrophilic interior and a hydrophobic exterior to facilitate the sample entering the capillary.

16. The method of claim 14, wherein the transferring the sample from the capillary into the analysis unit further comprises employing a low vacuum to aspirate the sample from the capillary into the analysis unit,
wherein the vacuum is also employed to stir the sample within the analysis unit.

17. The method of claim 14, wherein the sequence of coulometry and potentiometry is a time sequence comprising a coulometry on time, a coulometry off time, a potentiometry on time, and a potentiometry off time.

18. The method of claim 14, wherein one of the electrodes comprises silver and the target chemical comprises chloride, and
wherein the other electrode is pH sensitive and configured for current injection into the sample.

19. The method of claim 18, wherein the other electrode comprises gold, platinum, iridium, or palladium.
